(19) United States
(12) Patent Application Publication
(10) Pub. No.: US 2010/0256541 A1
(43) Pub. Date: Oct. 7, 2010

(54) Photo Therapy Apparatus Utilizing Embedded Optical Fibers

(75) Inventors: Brian Pryor, Newark, DE (US); Sean Xiaolu Wang, Wilmington, DE (US)

Correspondence Address: BWT PROPERTY, INC., 19 SHEA WAY, SUITE 301, NEWARK, DE, 19713, US

(73) Assignee: BWT PROPERTY, INC., Newark, DE (US)

(21) Appl. No.: 12/754,842

(22) Filed: Apr. 6, 2010

Related U.S. Application Data
(60) Provisional application No. 61/167,373, filed on Apr. 7, 2009.

(51) Int. Cl. A61F 5/01

(52) U.S. Cl. 602/2

(57) ABSTRACT

A photo therapy apparatus utilizing optical fibers embedded in a cast bandage. The optical fibers are used to deliver therapeutic light to the treatment area after the cast bandage is applied. The therapeutic light helps to decrease inflammation, pain, and irritation as well as to promote healing of the broken bones or sprained/strained muscles of the patient.

### REFERENCE TO RELATED APPLICATION

This application claims an invention which was disclosed in Provisional Patent Application No. 61/167,373, filed Apr. 7, 2009, entitled “PHOTO THERAPY APPARATUS UTILIZING EMBEDDED OPTICAL FIBERS”. The benefit under 35 USC §119(e) of the above mentioned United States Provisional Applications is hereby claimed, and the aforementioned application is hereby incorporated herein by reference.

### FIELD OF THE INVENTION

This invention generally relates to a photo therapy apparatus, and more specifically to a photo therapy apparatus utilizing embedded optical fibers.

### BACKGROUND

Orthopedic casts are used to hold a limb (or large portions of the body) with broken bones or sprained/strained muscles in place until healing is confirmed. Casts can be made of plaster or fiberglass bandages, which are molded over the injured body part and cured to hardness to provide support thereof.

The usage of cast can result in cutaneous complications such as macerations, ulcerations, infections, rashes, itching, burns, and allergic contact dermatitis. In hot weather, staphylococcal infection of the hair follicles and sweat glands can lead to severe and painful dermatitis.

To overcome the above cutaneous complications and speed up the healing of the broken bones or sprained/strained muscles, the present invention provides a photo therapy apparatus and method for treating the injured body part through optical fibers embedded in an orthopedic cast. The therapeutic light decreases inflammation/pain/irritation, kills bacteria, increases blood circulation, and promotes wound healing of the injured body part.

### SUMMARY OF THE INVENTION

A photo therapy apparatus for promoting the healing of broken bones or sprained/strained muscles of a patient, the photo therapy apparatus comprising: at least one light source to produce therapeutic light; a cast bandage applied conformably over a treatment area of the patient, said cast bandage is made substantially rigid after application to provide support to the treatment area;

and a plurality of optical fibers embedded in said cast bandage for delivering the therapeutic light from the light source to the treatment area to promote healing of the broken bones or the sprained/strained muscles.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 shows the first exemplary embodiment of the photo therapy apparatus;

FIG. 2 shows another variation of the photo therapy apparatus of FIG. 1;

FIG. 3 shows the second exemplary embodiment of the photo therapy apparatus.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a photo therapy apparatus utilizing embedded optical fibers. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

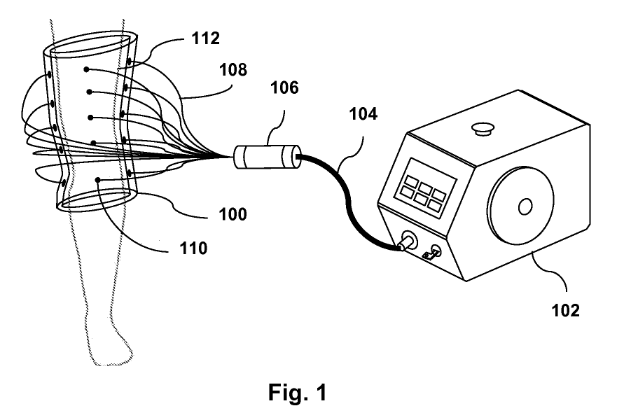

In the first exemplary embodiment of the present invention as shown in FIG. 1, a photo therapy apparatus is used to treat a patient with a broken limb. The photo therapy apparatus comprises a light source module 102, which may consist of lasers, light emitting diodes (LEDs), lamps or any of their combinations. The therapeutic light produced by the light source module 102 is coupled into an optical waveguide 104, such as an optical fiber or liquid light guide, and delivered to an output wand 106, where therapeutic light is coupled into a fiber bundle formed by the input ends of a plurality of optical fibers 108. The optical fibers 108 are embedded in a plaster or fiberglass cast 100 with their output ends 110 placed close to the skin 112 of the broken limb. When the cast 100 is cured to hardness to immobilize and hold the limb in a proper position, the output ends 110 of the fibers form a matrix of therapeutic points, which provide photo therapy to the skin 112 and the underneath muscle and bone tissue. The bundle of optical fibers 108 is detachable from the output wand 106 such that the optical fibers can be replaced every time when the cast is replaced. An optical diffuser (not shown) may be disposed between the skin 112 and the output ends 110 of the optical fibers to homogenize the intensity distribution of the output therapeutic light.

Depending on the output wavelength of the light source module, the photo therapy apparatus can provide a variety of therapeutic effects. The ultraviolet (UV)-blue light, at a wavelength of 370 to 490 nm, has high photon energy, which can help to produce singlet oxygen and effectively destroy bacteria. The red light, at a wavelength of 620 to 700 nm, can penetrate human tissue to a depth of about 8-10 mm. Skin layers, because of their high blood and water content, easily absorb red light, which helps to increase blood circulation and decrease inflammation/irritation. The near-infrared (NIR) light, at a wavelength of >700 nm, has been demonstrated to be beneficial for increasing cytochrome oxidase activity and ATP (adenosine triphosphate) content as well as promoting wound healing and relieving pain. In addition, the near-infrared light penetrates to a depth of about 30-40 mm, which makes it more effective for bones, joints, deep muscles, etc. In the present embodiment, the light source module 102 may comprise multiple light sources with different output wavelengths, where UV-blue and red light are employed to treat and prevent those cutaneous complications (e.g. macerations, ulcerations, infections, rashes, itching, burns, allergic contact dermatitis) that are caused by the usage of cast bandages, while NIR light is employed to speed up the healing of the broken bones or sprained/strained muscles.

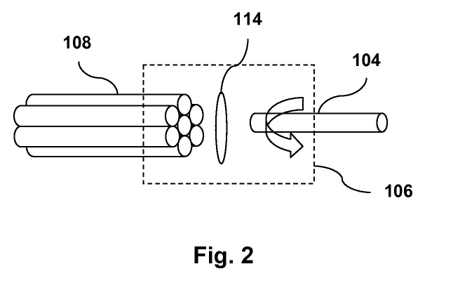

In another variation of the present embodiment, the therapeutic light from the optical waveguide 104 is coupled into only one or a few of the plurality of optical fibers 108 at a given time. This is achieved by a slight modification of the output wand 106 as shown in FIG. 2, where the position and focal length of the optical lens 114 (or any other kind of optical component used for fiber coupling) is controlled such that the image of the output end of the optical waveguide 104 falls on the input end of only one or a few of the plurality of optical fibers 108. By mechanically moving the optical waveguide 104, the optical fiber bundle 108, or the optical lens 114, the therapeutic light is sequentially delivered into the plurality of optical fibers 108, causing the output therapeutic light to scan over the skin of the broken limb. This scanning operation mode allows high optical power to be delivered to the subject tissue in a short period of time, thus avoiding any possible thermal damage.

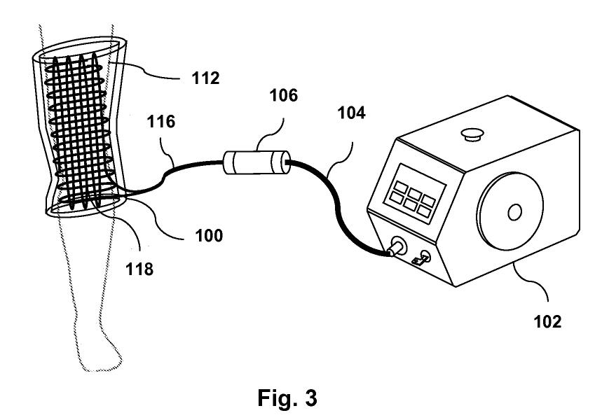

In the second exemplary embodiment of the present invention as shown in FIG. 3, the therapeutic light from the light source module 102 is delivered through an optical waveguide 104 and an output wand 106 into a plurality of side emitting optical fibers 116. The side emitting fibers are coiled or interlaced to form a pad-like structure 118, which is embedded in a plaster or fiberglass cast 100. When the cast 100 is cured to hardness, the optical fiber pad 118 is fixed in a position close to the skin 112 of the broken limb. The side-emitting fibers 116 comprise a roughened diffusive interface between their core and cladding region. The roughness of the diffusive interface is controlled such that a desired portion of the therapeutic light in the core region is refracted to emit from the side surface of the fiber. Similar therapeutic effects as described above can be produced in the subject tissue. The optical fiber pad 118 can be constructed with only a few of side emitting fibers, thus reducing the number of fiber connections.

In a slight variation of the present embodiment, the side emitting fiber based optical fiber pad can be replaced with a woven fiber-optic pad as disclosed in U.S. Pat. No. 5,339,223, which is hereby incorporated herein by reference. In this variation, standard end emitting optical fibers are interlaced to form a pad-like structure with a plurality of junctions. The micro-bending of the optical fiber at these junctions causes the therapeutic light to be emitted from the side surface of the fiber.

In all the above disclosed embodiments, the optical waveguide 104 can be omitted such that the therapeutic light from the light source 102 is directly coupled into the plurality of optical fibers 108 or 116. As an additional safety feature, temperature sensors can be incorporated at the output ends 110 or 118 of the optical fibers or in the cast material to monitor the temperature change of the subject tissue. The photo therapy method can be used with other types of cast bandages as well, such as those temporary casts or aircasts employing pneumatic braces.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A photo therapy apparatus for promoting the healing of broken bones or sprained/strained muscles of a patient, the photo therapy apparatus comprising:
at least one light source to produce therapeutic light;
a cast bandage applied conformably over a treatment area of the patient, said cast bandage is made substantially rigid after application to provide support to the treatment area; and
a plurality of optical fibers embedded in said cast bandage for delivering the therapeutic light from the light source to the treatment area to treat/prevent cutaneous complications and promote healing of the broken bones or the sprained/strained muscles.

2. The photo therapy apparatus of claim 1, wherein the plurality of optical fibers comprise at least one of side emitting optical fibers or end emitting optical fibers.

3. The photo therapy apparatus of claim 1, wherein the light source comprises at least one of lasers, light emitting diodes (LEDs), or lamps.

4. The photo therapy apparatus of claim 1, wherein the light source has an output wavelength in a range from ultraviolet (UV) to near infrared (NIR).

5. The photo therapy apparatus of claim 1, wherein the light source has multiple output wavelengths.

6. The photo therapy apparatus of claim 1, wherein the cast bandage comprises at least one of plaster bandages, fiberglass bandages, or aircast bandages.

7. The photo therapy apparatus of claim 1, further comprising at least one optical diffuser disposed at output ends of the plurality of optical fibers to homogenize the therapeutic light.

8. The photo therapy apparatus of claim 1, wherein the therapeutic light is simultaneously coupled into the plurality of optical fibers.

9. The photo therapy apparatus of claim 1, wherein the therapeutic light is sequentially coupled into the plurality of optical fibers, and wherein the therapeutic light is only coupled into a portion of the plurality of optical fibers at a given time.

10. The photo therapy apparatus of claim 1, further comprising at least one temperature sensor disposed at output ends of the plurality of optical fibers or in the cast bandage to monitor a temperature change of the treatment area.

11. A photo therapy method for promoting the healing of broken bones or sprained/strained muscles of a patient, the method comprising the steps of:
providing at least one light source to produce therapeutic light;
providing a cast bandage applied conformably over a treatment area of the patient, said cast bandage is made substantially rigid after application to provide support to the treatment area; and
providing a plurality of optical fibers embedded in said cast bandage for delivering the therapeutic light from the light source to the treatment area to treat/prevent cutaneous complications and promote healing of the broken bones or the sprained/strained muscles.

12. The photo therapy method of claim 11, wherein the plurality of optical fibers comprise at least one of side emitting optical fibers or end emitting optical fibers.

13. The photo therapy method of claim 11, wherein the light source comprises at least one of lasers, light emitting diodes (LEDs), or lamps.

14. The photo therapy method of claim 11, wherein the light source has an output wavelength in a range from ultraviolet (UV) to near infrared (NIR).

15. The photo therapy method of claim 11, wherein the light source has multiple output wavelengths.

16. The photo therapy method of claim 11, wherein the cast bandage comprises at least one of plaster bandages, fiberglass bandages, or aircast bandages.

17. The photo therapy method of claim 11, further comprising a step of providing at least one optical diffuser disposed at output ends of the plurality of optical fibers to homogenize the therapeutic light.

18. The photo therapy method of claim 11, wherein the therapeutic light is simultaneously coupled into the plurality of optical fibers.

19. The photo therapy method of claim 11, wherein the therapeutic light is sequentially coupled into the plurality of optical fibers, and wherein the therapeutic light is only coupled into a portion of the plurality of optical fibers at a given time.

20. The photo therapy method of claim 11, further comprising a step of providing at least one temperature sensor disposed at output ends of the plurality of optical fibers or in the cast bandage to monitor a temperature change of the treatment area.
